(19) United States
(12) Patent Application Publication
(10) Pub. No.: US 2021/0169701 A1
(43) Pub. Date: Jun. 10, 2021

(54) NANOMODIFIED TRANSFER DRAPE FOR EPIDERMAL GRAFTING

(71) Applicant: KCI Licensing, Inc., San Antonio, TX (US)

(72) Inventors: Diwi L. ALLEN, San Antonio, TX (US); James COURAGE, San Antonio, TX (US); Prathamesh KHARKAR, San Antonio, TX (US)

(21) Appl. No.: 16/768,614

(22) PCT Filed: Nov. 29, 2018
(86) PCT No.: PCT/US2018/062973

§ 371 Date: May 29, 2020

Related U.S. Application Data
(60) Provisional application No. 62/591,968, filed on Nov. 29, 2017.

(51) Int. Cl. A61F 13/02; A61L 15/32; A61L 15/24; A61L 15/58; A61B 17/322

(52) U.S. Cl. CPC A61F 13/02; A61L 15/325; A61F 2013/00157; A61L 15/58; A61B 17/322; A61L 15/24

(57) ABSTRACT

In one aspect, a dressing for transferring skin grafts from a donor site to a recipient site is disclosed, which comprises a substrate having a surface configured for contact with one or more epidermal skin grafts, a plurality of capture sites distributed across said substrate surface, wherein each of said capture sites is configured for capturing at least one epidermal skin graft, and a plurality of topographical features distributed over said substrate surface and coupled to at least one of said capture sites so as to provide at least one of inducing proliferation and facilitating migration of keratinocytes in the captured epidermal skin grafts to surrounding tissue.

### BACKGROUND

The present invention relates generally to dressings for transferring epidermal skin grafts from a donor site to a recipient site, and more particularly to such dressings that can facilitate the retention of the epidermal skin grafts and migration of keratinocytes in the grafts to surrounding tissue.

Skin grafting is a surgical procedure in which a section of skin is removed from one area of a person's body (autograft), removed from another human source (allograft), or removed from another animal (xenograft), and transplanted to a recipient site of a patient, such as a wound site. Chronic wounds are often observed in elderly patients and/or in patients with severe comorbidities. A common feature of different types of chronic wounds is impaired re-epithalization. Autologous epidermal grafting provides a solution to wound closure by transferring the patient's own epidermal cells to the wound site.

In one technique, a plurality of epidermal micrografts (also known as microdomes) are generated by raising a plurality of blisters through openings of an orifice plate attached to an end of a low-pressure chamber. The raised blisters can be harvested and transplanted onto a wound site. A device marketed by the assignee of the present application under the trade designation CelluTome® can be used to create and harvest such epidermal micrografts in an automated and precise manner.

Following transfer of the epidermal micrografts to a wound site, a medical professional needs to monitor re-epithalization of the site to ensure its proper healing. In some cases, such a medical professional may have difficulty differentiating between the microdomes and slough several days following the micrograft transfer and may mistakenly wipe away the micrograph outgrowth believing that the wound site contained slough. This can lead to a poor outcome.

Accordingly, there is a need for improved dressings for skin grafting.

### SUMMARY

In one aspect, a dressing for transferring skin grafts from a donor site to a recipient site is disclosed, which comprises a substrate having a surface configured for contact with one or more epidermal skin grafts, a plurality of capture sites distributed across said substrate surface, wherein each of said capture sites is configured for capturing at least one epidermal skin graft, and a plurality of topographical features distributed over said substrate surface and coupled to at least one of said capture sites so as to provide at least one of inducing proliferation and facilitating migration of keratinocytes in the captured epidermal skin grafts to surrounding tissue.

In some embodiments, the topographical features comprise a plurality of channels extending outwardly from said at least one capture site. In some embodiments, all capture sites are associated with one or more such channels. In some embodiments, the topographical features comprise a plurality of ridges extending outwardly from the capture sites.

In some embodiments, the capture sites include a plurality of wells configured for receiving one or more epidermal skin grafts. In other embodiments, the capture sites can include flat portions of the substrate surface to which an adhesive has been applied.

In some embodiments, at least one of the topographical features, e.g., at least one of a plurality of channels, can interconnect two or more of the capture sites, e.g., two or more of a plurality of wells provided on the substrate surface as capture sites.

In some embodiments, one or more of the skin-graft capture sites can include an adhesive for facilitating the capture of one or more skin grafts. In some such embodiments, the adhesive can exhibit a tackiness gradient extending from the center of a capture site to a periphery thereof. For example, the tackiness can decrease from the center of the capture site to its periphery.

In some embodiments, the adhesive can be applied to one or more capture sites as an adhesive film for at least partially coating at least one surface of the capture site(s) for facilitating the capture of epidermal skin grafts. By way of example, an adhesive film can be applied to the bottom surface of one or more wells provided on the substrate surface as capture sites. In some such embodiments, the adhesive film exhibits a thickness gradient characterized by a decreasing thickness from a center of a capture site, e.g., a well, to a periphery thereof.

In some embodiments, the adhesive film can exhibit a gradient in at least one chemical ingredient, which in turn imparts a tackiness gradient to the adhesive film.

In some embodiments, the adhesive film can have a surface density in a range of about 10 to about 200 grams/m², e.g., a surface density in a range of about 15 to about 90 grams/m².

By way of example, in some embodiments, the adhesive comprises a medical grade pressure-sensitive adhesive, such as a polyurethane adhesive, an acrylic adhesive, a high-tack silicone adhesive and a hydrocolloid-based adhesive.

In some embodiments, the capture sites, e.g., the wells, can have a width in a range of about 1 mm to about 5 mm, e.g., in a range of about 1 mm to about 4 mm or in a range of about 2 mm to about 3 mm. Further, in some embodiments, the topographical features can have a width in a range of about 100 nm to about 500 microns, e.g., in a range of about 200 nm to about 400 microns, or in a range of about 300 nm to about 300 microns.

In some embodiments in which the captures sites are in the form of a plurality of wells, the wells can have a depth in a range of about 1 micron to about 100 microns.

In some embodiments, at least one of the capture sites, e.g., the wells, and/or the topographical features can include a bioactive material. For example, the bioactive material can be applied to the bottom surface of at least one of the wells. In some such embodiments, the bioactive material can exhibit a concentration gradient along said at least one topographical feature. A variety of different bioactive materials can be employed. Some examples include, without limitation, collagen, keratinocyte growth factor (KGF), and epidermal growth factor (EGF).

In some embodiments, the substrate of a dressing according to the present teachings can include a plurality of perforations. In some such embodiments, the perforations can be distributed between the capture sites and the topographical features. By way of example, the perforations can have a width in a range of about 0.2 mm to about 2 mm, though other sizes can also be employed. In some embodiments, the perforations can have a cross-sectional area in a range of about 0.25 square millimeters to about 3 square millimeters. The perforations can have a variety of different cross-sectional shapes, such as polygonal, circular, etc.

A dressing according to the present teachings can be fabricated using fabrication techniques known in the art. For example, the topographical features can be formed via any of calendaring, film casting, extrusion, thermoforming during processing of a polymeric material to form said substrate. In some embodiments, the topographical features can be formed via any of chemical modulation, or embossing of said substrate surface.

In many embodiments, the substrate can be formed of a suitable polymeric material. Some examples of suitable polymeric materials include, without limitation, polyurethane, polypropylene, cellulosics, polyamides, polyvinyl alcohol, silicone elastomers, acrylics, and copolymers thereof.

In some embodiments, the substrate can have a thickness in a range of about 25 microns to about 200 microns.

In a related aspect, a method of transferring skin grafts from a donor site to a recipient site is disclosed, which comprises generating a plurality of epidermal skin blisters, placing a skin-graft contacting surface of a dressing on said epidermal skin blisters, where the dressing comprises a substrate having a surface configured for contact with one or more epidermal skin grafts, a plurality of capture sites distributed across said substrate surface, wherein each of said capture sites is configured for capturing at least one epidermal skin graft, and a plurality of topographical features distributed over said substrate surface and coupled to at least one of said capture sites so as to provide any of inducing proliferation and facilitating migration of keratinocytes in the captured epidermal skin grafts to surrounding tissue. The method further includes the step of cutting the epidermal skin blisters so as to capture said cut blisters at said capture sites of the transfer dressing.

In the above method, the dressing can have one or more of the properties discussed above. For example, in some embodiments of the above method, the capture sites can have a width in a range of about 1 mm to about 5 mm and the topographical features can have a width in a range of about 100 nm to about 500 microns. Further, the topographical features can include a plurality of channels extending outwardly from said at least one capture site. Further, at least one of the capture sites can include an adhesive. Moreover, at least one of the capture sites and/or the topographical features can include a bioactive material.

Further understanding of various aspects of the invention can be obtained by reference to the following detailed description in conjunction with the associated drawings, which are described briefly below.

### BRIEF DESCRIPTION OF THE DRAWINGS

FIG. 1A is a schematic, perspective view of a transfer dressing according to an embodiment of the present teachings,

FIG. 1B is a schematic top view of the dressing depicted in FIG. 1A,

FIG. 2 is a schematic cross-sectional view of the dressing depicted in FIGS. 1A and 1B,

FIG. 3 schematically depicts a capture site and associated topographical features extending outwardly from the capture site of the dressing depicted in the above figures, illustrating that the topographical features exhibit an increasing width from their proximal ends to their distal ends,

FIG. 4 is a schematic, perspective view of a transfer dressing according to another embodiment of the present teachings in which some of the topographical features connect a plurality of the capture sites,

FIG. 5 is a schematic, cross-sectional view of a transfer dressing according to another embodiment in which the skin-graft capture sites are in the form of flat portions of the substrate surface to which an adhesive has been applied,

FIG. 6 is a schematic, perspective view of a transfer dressing according to another embodiment in which the substrate includes a plurality of perforations between the capture sites and the topographical features,

FIG. 7 is a schematic top view of a capture sites and its associated topographical features to which a bioactive material has been applied,

FIG. 8 is a flow chart depicting various steps in a method according to the present teachings for transferring skin grafts from a donor site to a recipient site,

FIG. 9A is a schematic view of a device for generating a plurality of epidermal skin blisters, and

FIG. 9B shows a multi-plate cutter mechanism employed in the device of FIG. 8A.

### DETAILED DESCRIPTION

The present invention relates generally to a transfer dressing having a plurality of skin-graft capture sites, where the capture sites are associated with a plurality of topographical features, e.g., channels, that extend outwardly from the capture sites and can help induce proliferation and facilitate migration of keratinocytes in the capture epidermal skin grafts once the grafts are transferred to a recipient site. Various terms are used herein consistent with their ordinary meanings in the art.

The term “topographical feature,” as used herein, refers to a structure, such as a channel or a ridge provided on a substrate surface. The term “about,” as used herein, indicates a variation of at most 5% around a central value.

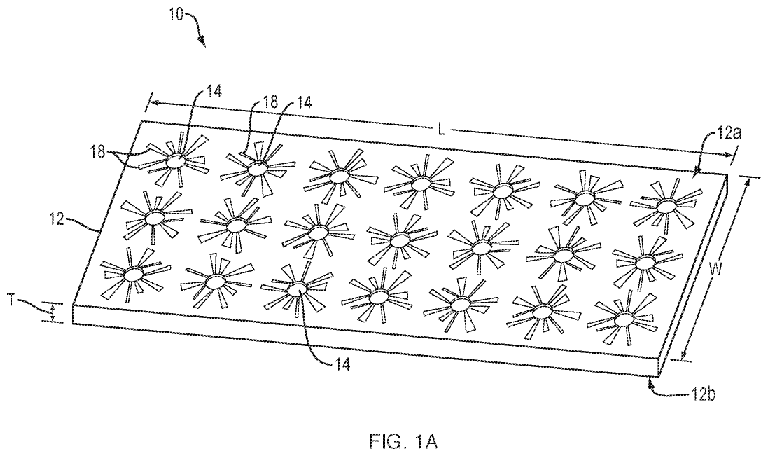

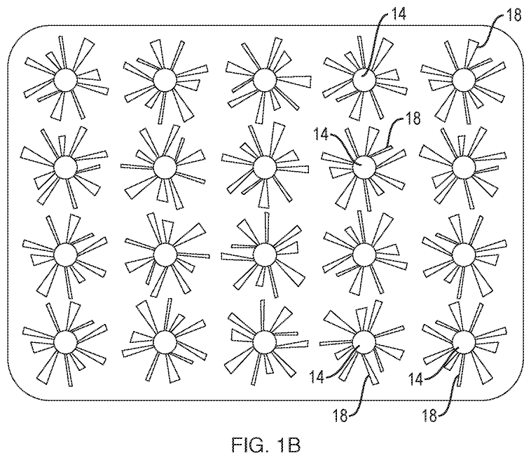

FIGS. 1A and 1B schematically depict a transfer dressing 10 (herein also referred to as a transfer drape) according to an embodiment that includes a substrate 12 having two opposed surfaces 12a and 12b, where the top surface 12a is configured for receiving epidermal skin grafts from a donor site. For example, as discussed in more detail below, the surface 12a can receive a plurality of epidermal blisters that were raised through the openings of an orifice plate positioned at the distal end of a vacuum chamber and cut via a cutting mechanism.

The substrate 12 can be formed of a variety of different materials. For example, in some embodiments, the substrate 12 can be formed of a suitable biocompatible polymeric material. By way of example, the substrate 12 can be formed of any of polyurethane, polyethylene, cellulosics, polyamides, polyvinyl alcohol, silicone, elastomers, acrylics, polypropylene, polyvinyl chloride and copolymers thereof.

The substrate 12 can have a variety of different sizes selected, for example, based on an intended application. By way of example, in some embodiments, the substrate 12 can have a width (W) in a range of about 5 cm to about 15 cm, and a length (L) in a range of about 5 cm to about 10 cm, though other sizes can also be employed. In some embodiments, the substrate 12 can have a thickness (T) in a range of about 25 micrometers (microns) to about 200 microns, though other thicknesses can also be employed.

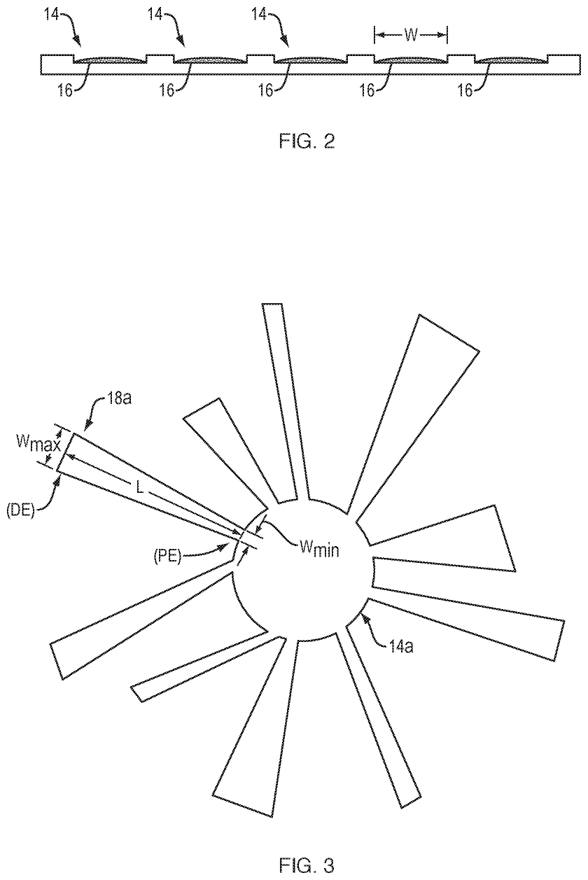

With continued reference to FIGS. 1A, 1B as well as FIG. 2, in this embodiment, the dressing 10 includes a plurality of skin-graft capture sites 14 distributed across the top surface 12a of the substrate 12. As discussed in more detail below, in this embodiment, the skin-graft capture sites 14 are in the form of a plurality of wells having an adhesive film 16 that at least partially coats the bottom surfaces of the wells so as to facilitate the capture of the skin-grafts by the wells.

More specifically, as shown schematically in FIG. 2, in this embodiment a thin film of adhesive 16 that is disposed on the bottom surface of the wells 14 exhibits a decreasing thickness from the center of the well to a periphery thereof.

Although in this embodiment the wells 14 have flat bottom surface, in other embodiments, they can have different shapes, e.g., a hemispherical shape. In this embodiment, the wells 14 can have a width (w) in a range of about 1 mm to about 5 mm, and a depth in a range of about 1 micron to about 100 microns, though other sizes can also be employed.

In this embodiment, the wells 14 are distributed as a regular array across the top surface 12a of the substrate 12. By way of example, in some applications, the spacing between the wells 14 can be selected such that the wells will be in substantial register with a plurality of blisters formed through the orifice plate of a cellutome® device when the substrate surface 12a is placed over the blisters. This will allow each of the wells to capture a corresponding blister when the blisters are cut.

Referring again to FIGS. 1A and 1B, the dressing 10 further includes a plurality of topographical features 18 distributed across the skin-graft receiving surface 12a of the substrate 12. In this embodiment, the topographical features 18 are in the form of a plurality of channels that extend radially outwardly from the wells 14.

As shown schematically in FIG. 3, a proximal end (PE) of each channel 18 (e g , channel 18a depicted in this figure) is coupled to one of the wells 14 (e.g., well 14a depicted in this figure). The channel 18a terminates in a distal end (DE) at a length (L), which can be, for example, in a range of about 0.5 mm to about 10 mm, from the proximal end. In this embodiment, the channels 18 exhibit a varying width, which increases from the proximal end of each channel (i.e., the end coupled to one of the wells) to its distal end. By way of example, the illustrative channel 18a has a minimum width (Wₘₘ), e.g., 10 nm, at its proximal end and a maximum width (Wₘₐₓ) at its distal end. The transition from the minimum width (W_{m.}) to its maximum width (Wₘₐₓ) can be linear or non-linear. By way of example, in some embodiments, the minimum width (Wₘ) can be in a range of about 50 nm to about 100 nm and the maximum width (Wₘₐₓ) can be in a range of about 400 microns to about 500 microns, though other sizes can also be employed, e.g., based on an intended application of the dressing.

As noted above, the bottom of the wells 14 can be coated with an adhesive film, which can facilitate the capture of epidermal skin grafts within the wells. In many embodiments, the channels 18 are not, however, coated with an adhesive film so as to not inhibit the migration of cells along the channels. For example, the adhesive film disposed at the bottom of the wells 14 can exhibit a decreasing thickness from the center of the wells to their periphery such that the channels are substantially free of such adhesive film.

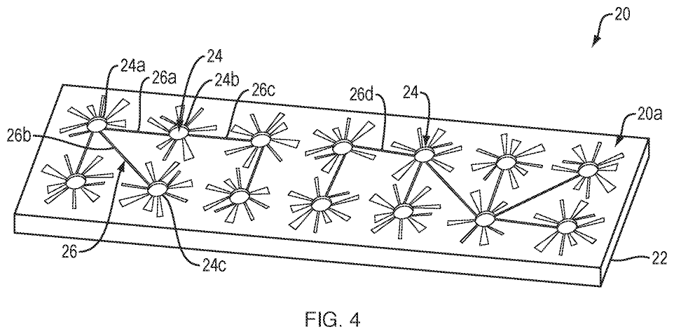

In some embodiments, at least some of the topographical features disposed on the skin-graft receiving surface of the substrate interconnect two or more of the capture sites. By way of example, FIG. 4 schematically depicts a dressing 20 according to such an embodiment, which includes a substrate 22 having a skin-graft receiving surface 20a on which a plurality of skin-graft capture sites 24, e.g., in the form of a plurality of wells, such as the wells discussed above with regard to the transfer dressing 10, are distributed. The illustrative dressing 20 further includes a plurality of channels 26 that extend outwardly from the skin-graft capture sites 24. In this embodiment, a plurality of the channels 26, i e, channels 26a, 26b, 26c, and 26d provide pairwise connections between some of the wells, e.g., the nanostructured channel 26a connects the well 24a to the well 24b and the channel 26b connects the well 24a to the well 24c. In other embodiments, more of the channels 26, and in some cases all of the channels 26, can provide pairwise interconnections between the skin-graft capture wells 24. The interconnection of at least some of the wells via the channels can facilitate the migration of the epidermal grafts between the wells. In some embodiments, this can in turn induce a faster rate of wound coverage that could be more visible to a caregiver assessing the progress of the treatment.

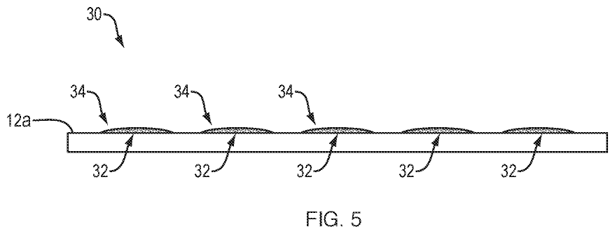

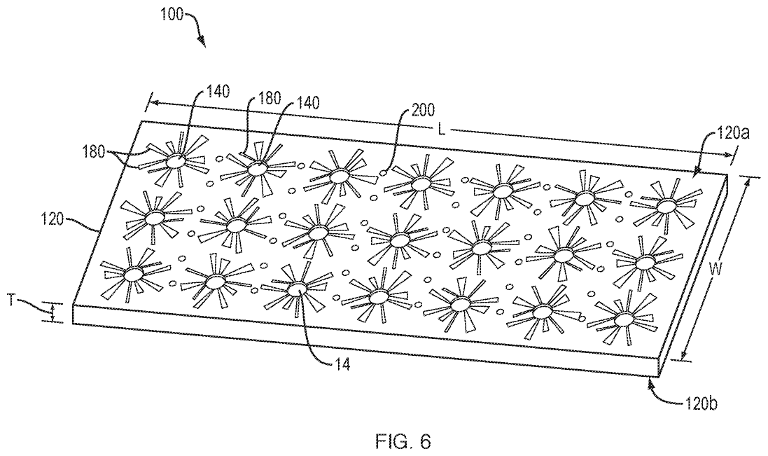

In some embodiments, the skin-graft capture sites can be flat portions of the top surface 12a of the substrate 12. By way of example, FIG. 5 schematically depicts such a dressing 30, which includes a plurality of substantially flat capture sites 32 to each of which an adhesive 34 is applied to facilitate the capture of a plurality of epidermal skin grafts. Although in this illustrative embodiment, the adhesive films 34 are depicted as having a uniform thickness across each capture site, in other embodiments the thickness of the adhesive films 34 can exhibit a gradient characterized by a greater thickness at the center of the wells and a lower thickness at the periphery. Further, similar to the previous embodiments, the dressing 30 includes a plurality of topographical features (not shown in this figure) in the form of channels that radiate outwardly from the capture sites.

In some embodiments, the substrate of a transfer dressing according to the present teachings can include a plurality of perforations distributed between the capture sites and their associated topographical features. By way of example, FIG. 6 schematically depicts a transfer dressing 100 according to another embodiment, which includes a perforated substrate 120 having a skin-contacting surface 120a and an opposed surface 120b. Similar to the transfer dressing depicted in FIGS. 1A and 1B, the dressing 100 includes a plurality of skin-graft captures sites 140 and a plurality of topographical features 180 extending from the skin-graft capture sites. A plurality of perforations in the form of cylindrically-shaped holes 200 are distributed in the substrate 100. In some embodiments, the holes 200 can have a diameter in a range of about 0.2 mm to 2 mm. In some embodiments, the holes 200 can have a cross-sectional area in a range of about 0.25 mm² to about 3 mm². In other embodiments, the perforations can have a polygonal cross-sectional shape. In some embodiments, the holes 200 extend from the surface 120a to the opposed surface 120b. In other embodiments, one or more of the holes 200 have openings at the surface 120a and extend partially into the substrate (that is, they do not extend all the way to the opposed surface 120b). The perforations 200 can advantageously facilitate the exudate drainage at the recipient site (e.g., a wound).

As discussed in more detail below, a dressing according to the present teachings, such as dressings 10, 20 and 30 can provide a number of advantages. For example, the skin-graft capture sites can confine the captured microdomes and the topographical features can concurrently present topographic cues to the keratinocytes/primary human cells, thereby guiding and facilitating their migration.

Moreover, once the dressing is placed on a recipient site, the sandwich-type transplants (i.e., the harvested microdomes sandwiched between the transplant site and the dressing) may result in an even distribution of microskin grafts, greatly improving the “take” rate of microskin tissue, thereby promoting re-epithalization. In addition, the void area in the functionalized dressing, i.e., the area of the dressing that does not contain topographical structures, can be suitable for exudate drainage in wound.

Moreover, the pattern of wells and topographical features can help a clinician distinguish between re-epithalized tissue and slough, thus inhibiting the removal of re-epithalized tissue by mistake.

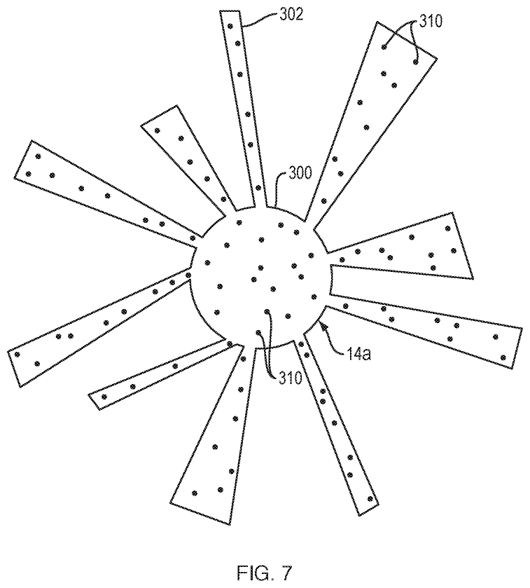

In some embodiments, a bioactive material can be disposed in one or more of the capture sites and/or the topographical features. By way of example, FIG. 7 schematically depicts a capture site 300 and its associated topographical features 302, where a bioactive material 310 is applied to the capture site 300 and a plurality of its associated topographical features 302. A variety of different bioactive materials can be employed. Some examples include, without limitation, collagen, keratinocyte growth factor (KGF), and epidermal growth factor (EGF).

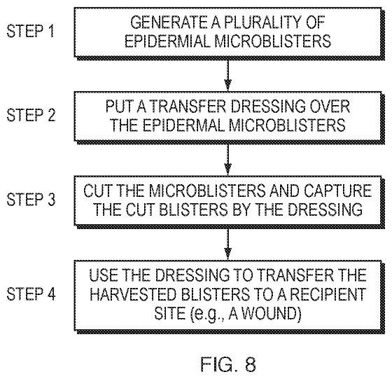

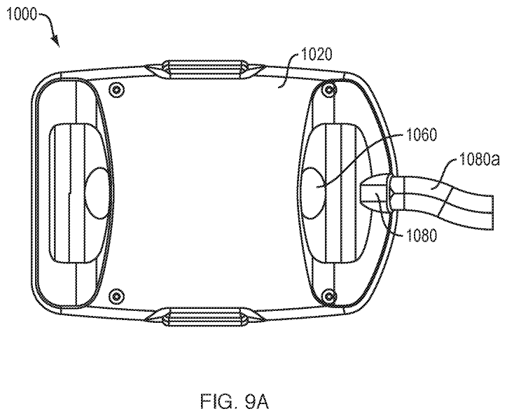

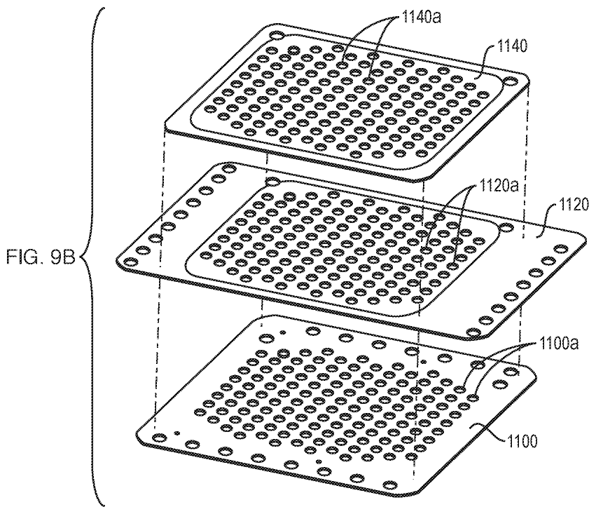

With reference to the flow chart of FIG. 8, in one embodiment, a plurality of epidermal microblisters can be formed. By way of example, a cellutome® device can be employed to form such epidermal microblisters. For example, with reference to FIGS. 9A and 9B, such a device 1000 can include a head 1020 that can be removably and replaceably attached to a harvester (not visible in this figure) to form a hollow chamber 1060 into which a plurality of blisters can be drawn. In particular, the head 1020 can include a suction coupling 1080 that allows coupling the head via a suction tubing 108a to a vacuum source (not shown) to generate a negative pressure within the chamber. The harvester includes an orifice plate 1100 having a plurality of openings 1100a through which a plurality of epidermal microblisters can be raised into the hollow chamber 1060. The harvester further includes a cutter plate 1120 disposed between the orifice plate 1100 and an upper plate 1140. Both the cutter plate 1120 and the upper plate 1140 include a plurality of openings 1120a and 1140a, respectively, through which the blisters can protrude. Once the blisters are formed, the cutter plate 1120 can be moved relative to the orifice plate and the upper plate to cut the blisters. Further details regarding such a blister-generating device can be found, e.g., in U.S. Pat. No. 9,173,674 titled “Devices for Harvesting a Skin Graft,” which is herein incorporated by reference in its entirety.

More specifically, once the blisters are formed, the application of reduced pressure to the chamber 1060 can be discontinued, and the head portion 1020 can be removed to expose the epidermal blisters. A dressing according to the present teachings can then be placed over the epidermal blisters (step 2 in the flow chart of FIG. 7). In some embodiments, the number of the capture sites provided on the dressing is equal to the number of the formed epidermal blisters and the distribution of the capture sites across the skin-graft receiving surface of the dressing is such that there is a one-to-one correspondence between the capture sites and the epidermal blisters.

The epidermal blisters can then be cut and the cut blisters can be captured by the skin-graft capture sites of the dressing (step 3 in the flow chart of FIG. 7). The dressing can then be placed on a recipient site, e.g., a wound site, to transfer the harvested blisters to a recipient site, e.g., a wound, so as to promote re-epithalization of the recipient site.

In some embodiments, the topographical features can be generated by means of thermoforming, film casting, calendaring, or a combination of these techniques. In another embodiment the topographical features can be created post-polymer processing by means of chemical modulation and/or embossing. By way of example, a thin layer of pressure sensitive adhesive can be coated onto the substrate to maintain the topographical features. In addition, the topographical features can be introduced on a pressure-sensitive adhesive coated polymeric film by means of embossing or engraving (positive or negative)/rotogravure process.

Those having ordinary skill in the art will appreciate that various changes can be made to the above embodiments without departing from the scope of the invention.

1. A dressing for transferring skin grafts from a donor site to a recipient site, the dressing comprising:
a substrate having a substrate surface configured to contact a plurality of epidermal skin grafts;
a plurality of capture sites distributed across said substrate surface, the plurality of capture sites being configured to capture at least one epidermal skin graft; and
a plurality of topographical features distributed over said substrate surface, the plurality of topographical features being coupled to at least one of said plurality of capture sites and configured to provide at least one of: inducing proliferation and facilitating migration of keratinocytes in the at least one epidermal skin graft to surrounding tissue.

2. The dressing of claim 1, wherein said plurality of topographical features comprise a plurality of channels extending outwardly from said at least one of the plurality of capture sites.

3. The dressing of claim 2, wherein said at least one of the plurality of capture sites comprises a plurality of wells configured to receive the at least one epidermal skin graft.

4. The dressing of claim 3, wherein at least some of said plurality of channels interconnect two or more of said plurality of wells.

5. The dressing of claim 1, wherein said plurality of capture sites comprise an adhesive configured to facilitate capture of said at least one epidermal skin grafts.

6. The dressing of claim 5, wherein said adhesive comprises at least one of: a tackiness gradient from a center of at least one of said plurality of capture sites to a periphery thereof, a tackiness configured to decrease from the center of the at least one of the plurality of capture sites to the periphery thereof, and a gradient in at least one chemical composition that imparts said tackiness gradient thereto.

7. (canceled)

8. The dressing of claim 3, further comprising an adhesive film configured to at least partially coat a surface of at least one of the plurality of wells to facilitate capture of the at least one epidermal skin graft, wherein the adhesive film comprises a thickness gradient characterized by a decreasing thickness from a center of the well to a periphery thereof.

9. (canceled)

10. (canceled)

11. The dressing of claim 8, wherein said adhesive film comprises at least one of: a surface density in a range of about 10 to about 200 grams/m², a surface density in a range of about 15 to about 90 grams/m².

12. (canceled)

13. The dressing of claim 5, wherein said adhesive comprises at least one of: a medical grade pressure-sensitive adhesive, a polyurethane adhesive, an acrylic adhesive, a high-tack silicone adhesive, and a hydrocolloid-based adhesive.

14. (canceled)

15. The dressing of claim 1, wherein said capture sites comprise at least one of: a width in a range of about 1 mm to about 5 mm, a width in a range of about 1 mm to about 4 mm, and a width in a range of about 2 mm to about 3 mm.

16. (canceled)

17. (canceled)

18. The dressing of claim 1, wherein said plurality of topographical features comprises at least one of: a width in a range of about 100 nm to about 500 microns, a width in a range of about 200 nm to about 400 microns, and a width in a range of about 300 nm to about 300 microns.

19. (canceled)

20. (canceled)

21. The dressing of claim 3, wherein said plurality of wells comprise at least one of: a width in a range of about 1 mm to about 5 mm, a width in a range of about 1 mm to about 4 mm, a width in a range of about 1 mm to about 3 mm, a width in a range of about 1 mm to about 2 mm, and a depth in a range of about 1 micron to about 100 microns.

22-25. (canceled)

26. The dressing of claim 1, further comprising a bioactive material disposed in at least one of said plurality of topographical features, the bioactive material comprising a concentration gradient along said at least one topographical feature.

27. (canceled)

28. The dressing of claim 26, wherein said bioactive material comprises at least one of: collagen, keratinocyte growth factor (KGF), and epidermal growth factor (EGF).

29. The dressing of claim 1, wherein the at least one of said plurality of capture sites comprises at least one bioactive material and the at least one bioactive material comprises at least one of: collagen, keratinocyte growth factor (KGF), and epidermal growth factor (EGF).

30. (canceled)

31. The dressing of claim 1, wherein said substrate comprises a plurality of perforations dispersed between the plurality of capture sites and the plurality of topographical features.

32. The dressing of claim 31, wherein the plurality of perforations comprise at least one of: a width in a range of about 0.2 mm to about 2 mm, a cross-sectional area in a range of about 0.25 square millimeters to about 3 square millimeters, a polygonal cross-sectional shape, and a circular cross-sectional shape.

33-36. (canceled)

37. The dressing of claim 1, wherein said plurality of topographical features are formed via at least one of: calendaring, film casting, extrusion, thermoforming during processing of a polymeric material to form said substrate, chemical modulation, and embossing of said substrate surface.

38. (canceled)

39. The dressing of claim 1, wherein said substrate comprises a polymeric material, and the polymeric material comprises at least one of: polyurethane, polypropylene, cellulosics, polyamides, polyvinyl alcohol, silicone elastomers, acrylics, and copolymers thereof.

40. (canceled)

41. The dressing of claim 1, wherein said substrate comprises a thickness in a range of about 25 microns to about 200 microns.

42. A method of transferring skin grafts from a donor site to a recipient site, the method comprising:
generating a plurality of epidermal skin blisters;
placing a skin-graft contacting surface of a transfer dressing on said epidermal skin blisters, said transfer dressing comprising:
a substrate surface configured to contact one or more epidermal skin grafts;
a plurality of capture sites distributed across said substrate surface, the plurality of capture sites being configured to capture at least one epidermal skin graft; and
a plurality of topographical features distributed over said substrate surface, the plurality of topographical features being coupled to at least one of said plurality of capture sites and configured to provide at least one of: inducing proliferation and facilitating migration of keratinocytes in the at least one epidermal skin graft to surrounding tissue; and
cutting said plurality of epidermal skin blisters and capture cut blisters on said plurality of capture sites of the transfer dressing.

43-47. (canceled)
